(19) United States
(12) Patent Application Publication
(10) Pub. No.: US 2024/0277087 A1
(43) Pub. Date: Aug. 22, 2024

(54) FUNCTIONAL MASK

(71) Applicants: Vijay K. Dhir, Santa Monica, CA (US); Shuaihang Pan, Los Angeles, CA (US); Keshiv S. Tandon, Los Altos Hills, CA (US); Jasmine Li, Manhattan Beach, CA (US); THE REGENTS OF THE UNIVERSITY OF CALIFORNIA, Oakland, CA (US)

(72) Inventors: Vijay K. Dhir, Santa Monica, CA (US); Shuaihang Pan, Los Angeles, CA (US); Keshiv S. Tandon, Los Altos Hills, CA (US); Jasmine Li, Manhattan Beach, CA (US)

(21) Appl. No.: 18/571,863

(22) PCT Filed: Mar. 3, 2022
(86) PCT No.: PCT/US22/18731

§ 371 Date: Dec. 19, 2023

Related U.S. Application Data
(60) Provisional application No. 63/216,224, filed on Jun. 29, 2021.

(51) Int. Cl. A41D 13/11; A61L 2/00

(52) U.S. Cl. CPC A41D 13/1192; A61L 2/0023; A61L 2202/16; A61L 2202/21

(57) ABSTRACT

The present invention provides a face mask having at least one heating element configured to inactivate microorganisms including but not limited to bacteria, fungi, yeast, molds, viruses, etc.

### CROSS-REFERENCE TO RELATED APPLICATIONS

This application claims priority to U.S. Provisional Patent Application No. 63/216,224 filed Jun. 29, 2021, the contents of which are incorporated by reference herein in its entirety.

### BACKGROUND OF THE INVENTION

Recently, there has been great interest in different ways to reduce the risk of infection not only in nursing homes, hospitals, and hospices throughout the nation, but also in the doctor's and dentist's office, as well as in non-healthcare settings such as businesses, offices, schools, universities, and other places where people congregate. The healthcare and non-healthcare environments contain a diverse population of microorganisms, which can cause infection. Microorganisms (e.g., bacteria, fungi, yeast, molds, and viruses) in air and water, on surfaces, on skin, in bodily fluid (e.g., blood, saliva, secretions, wound exudate, etc.), breath and other sources tend to be the biggest players in the spread of infection. Not only are patients at risk of developing infection, but also are the visitors, nurses, doctors, or other healthcare and non-healthcare workers and general public that come into contact with these infectious sources.

Protective masks have been widely used by personnel in hospitals, researchers in laboratories, workers in construction sites, as well as the general public in highly polluted areas or during flu season. Most filtering barriers of the conventional protective masks are not functionalized with biocides or virucides. Therefore, those protective masks simply serve as a physical barrier to filter out contaminants. When it comes to viruses and bacteria, those barriers cannot kill them on the spot. The ability to kill bacteria and/or viruses on the spot is a desirable function for protective masks. Further, when wearing a mask, a problem arises, which is the risk of a secondary infection, since viruses entering the mask used may be in the hands after removing it. Therefore, the mask is not sufficiently effective for the complete prevention of infections.

Thus, there is a need in the art for a functional mask capable of effectively protecting the wearer by killing bacteria and/or viruses on the spot. The present invention meets this need.

### SUMMARY OF THE INVENTION

In one aspect, the present invention provides a face mask comprising: an outer layer; an inner layer; and at least one heating element positioned between the outer layer and the inner layer. In one embodiment, the mask further comprises one or more middle layers positioned between the outer layer and the inner layer. In one embodiment, wherein the outer layer further comprises a resealable slot or fold opening configured to allow insertion and removal of the at least one heating element. In one embodiment, the inner layer further comprises a resealable slot or fold opening configured to allow insertion and removal of the at least one heating element. In one embodiment, wherein the inner layer and the outer layer both comprise a resealable slot or fold opening configured to allow insertion and removal of the at least one heating element. In one embodiment, the mask is configured to cover a wearer's nasal and oral region. In one embodiment, one or more heating element is configured to heat up to a temperature ranging between approximately 40-50° C. In one embodiment, the at least one heating element further comprise a controller configured to initiate or terminate heating of at least one heating element. In one embodiment, the at least one heating element is removable. In one embodiment, the at least one heating element is powered by a battery. In one embodiment, the at least one heating element is powered by a rechargeable source. In one embodiment, the rechargeable source is positioned outside the mask and is carried by a wearer. In one embodiment, the at least one heating element are configured to allow passage of air through the at least one heating element to the inner layer and to a wearer. In one embodiment, the at least one heating element are further be covered by a protective insulating layer. In one embodiment, the at least one heating element is an electrical heating element. In one embodiment, the at least one heating element has a plurality of channels that allows air passage therethrough and wherein the plurality of channels have a parallel structure. In one embodiment, the mask further comprises at least one strap configured to secure the mask to a wearer's face. In one embodiment, the mask further comprises a deformable member, configured to be bent or pressed by a wearer into a configuration that allows the body of the mask to conform to the wearer's face. In one embodiment, the mask further comprise a breathing zone positioned directly in front of the nasal and oral region. In one embodiment, the at least one heating element is positioned within the breathing zone.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts an exemplary functional mask of the present invention.

FIG. 2 depicts a front and back view of an exemplary functional mask of the present invention.

FIG. 3 depicts another exemplary functional mask of the present invention.

FIG. 4 depicts the front view of an exemplary functional mask of the present invention connected to a rechargeable battery pack.

FIG. 5 depicts the back view of an exemplary functional mask of the present invention connected to a rechargeable battery pack.

FIG. 6 depicts an experimental setup with heating elements housed in a petri dish, inoculated with SARS-COV-2 (red arrow points to viral smear).

FIG. 7 depicts an experimental setup with SARS-COV-2 viral solution placed under the heating element on a petri dish.

FIG. 8 depicts results of experiments investigating SARS-COV-2 after heat treatment for 1 hour.

FIG. 9 depicts results of experiments investigating virus viability in presence of the heating element.

FIG. 10 depicts results of experiments investigating percent inhibition of the viruses in presence of the heating element.

FIG. 11 depicts results of experiments investigating the temperature rise of the heating element in the functional mask.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of functional masks. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate. The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Functional Mask

The present invention provides a face mask configured to inactivate microorganisms including but not limited to bacteria, fungi, yeast, molds, viruses, etc. In one embodiment, the face mask of the present invention is configured to inactivate viruses including but not limited to SARS-COV-2. In one embodiment, the face mask of the present invention is configured to provide filtered air from the surrounding environment to the user.

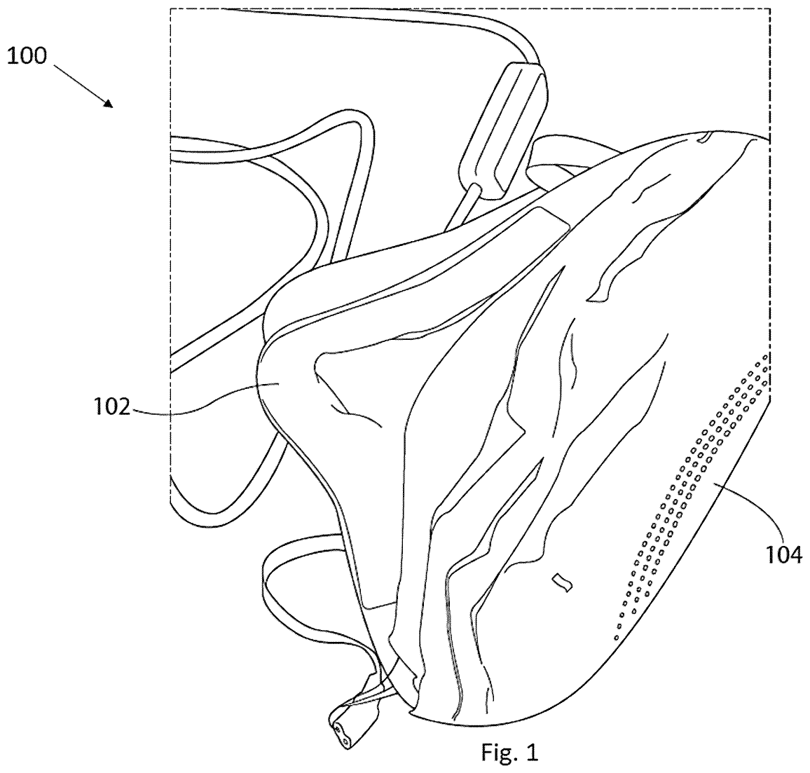

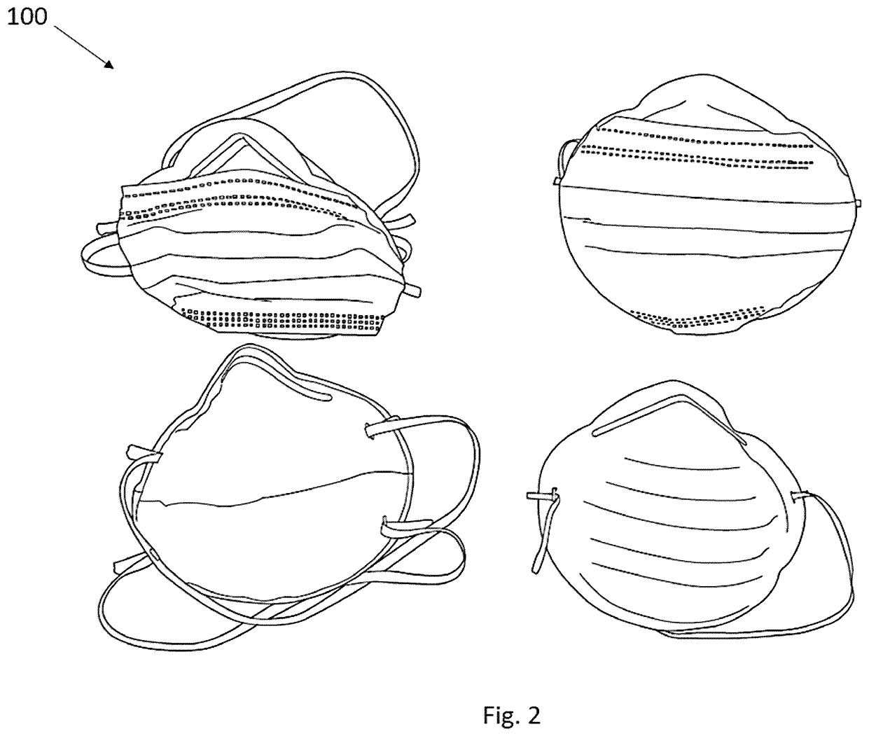

Referring now to FIG. 1 and FIG. 2, an exemplary mask 100 of the present invention is shown. Mask 100 comprises an outer layer 102, an inner layer 104, at least one heating element 106.

Outer layer 102 is positioned farthest from a wearer's face and inner layer 104 is closest to the wearer's face. In one embodiment, mask 100 is configured to be positioned on the wearer's face and cover their nasal and oral region. In one embodiment, outer layer 102 and inner layer 104 may be made from a filtering material including but not limited to a sheet, a cloth, a film, a web, a woven material, a non-woven material, and combinations thereof. In one embodiment, the non-woven materials may include but are not limited to wet laid fibers, dry laid fibers, spun-laced fibers, spun-bond fibers, melt-blown fibers, spunbonded-melt blown-spunbonded (SMS) fibers, carded fibers, thermoplastic fibers, regenerated fibers, and bicomponent fibers such as sheath-core fibers. These non-woven materials may include, but are not limited to, polyolefins such as polyethylene and polypropylene, polyesters such as PET, natural fibers, and cellulose materials. In one embodiment, the non-woven materials forming outer layer 102 and/or inner layer 104 may comprise mixtures of two or more of the foregoing fiber types. In one embodiment, outer layer 102 and inner layer 104 may comprise one or more webs of fine inorganic fibers or polymeric synthetic fibers. In one embodiment, outer layer 102 and inner layer 104 may comprise melt-blown fabrics. In one embodiment, the melt-blown fabrics adopt an electrostatic electret treatment process using an electret master-batch to increase the electrostatic adsorption area of the melt blown cloth while reducing its ventilation resistance. This allows for an increase in mask 100 filtration capacity, thus extending the life of the protective mask.

In one embodiment, mask 100 may further comprise at least one middle layer. In one embodiment, at least one middle layer may be made from a filtering material including but not limited to a sheet, a cloth, a film, a web, a woven material, a non-woven material, and combinations thereof. In one embodiment, the non-woven materials may include but are not limited to wet laid fibers, dry laid fibers, spun-laced fibers, spun-bond fibers, melt-blown fibers, spunbonded-melt blown-spunbonded (SMS) fibers, carded fibers, thermoplastic fibers, regenerated fibers, and bicomponent fibers such as sheath-core fibers. These non-woven materials may include, but are not limited to, polyolefins such as polyethylene and polypropylene, polyesters such as PET, natural fibers, and cellulose materials. In one embodiment, the non-woven materials forming at least one middle layer may comprise mixtures of two or more of the foregoing fiber types.

In one embodiment, at least one middle layer may comprise one or more webs of fine inorganic fibers or polymeric synthetic fibers. In one embodiment, at least one middle layer may comprise melt-blown fabrics. In one embodiment, the melt-blown fabrics adopt an electrostatic electret treatment process using an electret master-batch to increase the electrostatic adsorption area of the melt blown cloth while reducing its ventilation resistance. This allows for an increase in mask 100 filtration capacity, thus extending the life of the protective mask.

In one embodiment, at least one middle layer may have a cup-shaped configuration. In one embodiment, at least one middle layer may have a flat-shaped configuration. In one embodiment, at least one middle layer may have any other shapes known to one skilled in the art.

In one embodiment, outer layer 102, inner layer 104 and at least one middle layer may be made from the same material. In one embodiment, outer layer 102, inner layer 104 and at least one middle layer may be made from different materials. In one embodiment, outer layer 102, inner layer 104 and at least one middle layer may be affixed to one another along their respective outer edges. In one embodiment, outer layer 102, inner layer 104 and at least one middle layer may be affixed to one another by any one or combination of sewing, gluing, heat sealing, welding, ultrasonic bonding and/or any other suitable bonding procedure.

In one embodiment, outer layer 102 and inner layer 104 may have a cup-shaped configuration. In one embodiment, outer layer 102 and inner layer 104 may have a cone-shaped configuration. In one embodiment, outer layer 102 and inner layer 104 may have a flat-shaped configuration. In one embodiment, outer layer 102 and inner layer 104 may have any other shapes known to one skilled in the art.

In one embodiment, outer layer 102 and inner layer 104 may comprise at least one pleat configured to allow for expansion of the mask, so it can cover the face of the wearer from the nose to under their chin. In one embodiment, the pleats may be formed by folding the material and joining the sides of the body of outer layer 102 or inner layer 104 by any available method including, but not limited to, stitching, ultrasonic welding, heat welding, etc. to hold the pleats in place.

In one embodiment, mask 100 may further comprise at least one sealing member. In one embodiment, the at least one sealing member may have a uniformly wide shape, including but not limited to a rectangular, circular, oval, square shape. In one embodiment, the at least one sealing member may be narrower at a center section of mask 100, corresponding to the bridge of the nose. In one embodiment, the at least one sealing member may be made of any material that is flexible so that it can conform to the contours of a wearer's face. In one embodiment, the at least one sealing member may be made of a material that exhibits low water absorption and vapor transmission.

In one embodiment, the at least one sealing member may be integral with outer layer 102 or inner layer 104. In one embodiment, the at least one sealing member may be attached to outer layer 102 or inner layer 104. In one embodiment, the at least one sealing member may be joined to outer layer 102 or inner layer 104 by any possible method including, but not limited to, stitching, heat welding, ultrasonic welding, and adhesive bonding.

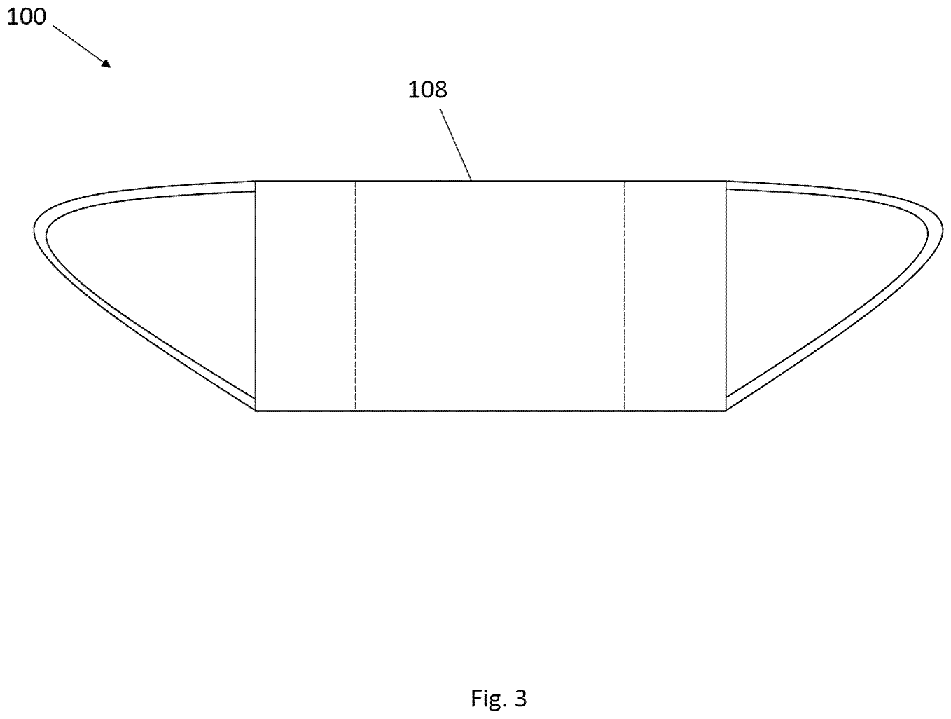

Referring now to FIG. 3, in one embodiment, mask 100 may further comprise a breathing zone 108. Breathing zone 108 may be positioned directly in front of the nasal and oral region. In one embodiment, breathing zone 108 may have any suitable shape known to one skilled in the art including but not limited to rectangular, circular, square, etc. In one embodiment, breathing zone 108 may be any size or percentage of mask 100, known to one skilled in the art.

At least one heating element 106 may be positioned between outer layer 102 and inner layer 104. In one embodiment, at least one heating element 106 may be integrated within the at least one middle layer. In one embodiment, at least one heating element 106 may be positioned directly in front of inner layer 104, inside the mask. In one embodiment, at least one heating element 106 may be positioned directly behind outer layer 102. In one embodiment, at least one heating element 106 may be positioned in breathing zone 108. In one embodiment, at least one heating element 106 may be positioned to either side of beathing zone 108. In one embodiment, a portion of at least one heating element 106 may be positioned in breathing zone 108.

In one embodiment, at least one heating element 106 is able to provide a temperature ranging between 10-100° C. In one embodiment, at least one heating element 106 is able to provide a temperature ranging between 40-50° C. In one embodiment, at least one heating element 106 may be configured to heat up to a single, consistent temperature. In one embodiment, at least one heating element 106 may have a configuration capable of allowing air to pass through and then enter the wearer's nose and/or mouth, while the heat inactivates microorganisms that passed through outer layer 102. In one embodiment, at least one heating element 106 may have plurality of channels that allows air passage therethrough. In one exemplary embodiment, at least one heating element 106 may have any channel structure known to one skilled in the art including but not limited to a parallel structure, a honeycomb structure, etc.

In one embodiment, at least one heating element 106 may be an electrical heating element. In one embodiment, at least one heating element 106 may include one or more electrically conductive wires (e.g., copper wire, aluminum wire, tin wire, steel wire, etc.). In one embodiment, at least one heating element 106 may be made from any material known to one skilled in the art including but not limited to a metal, ceramic, etc. In one embodiment, at least one heating element 106 may be made from any high electrical resistance material known to one skilled in the art including but not limited to nichrome, platinum, etc. In one embodiment, at least one heating element 106 may be any known means of providing heat known to one skilled in the art.

In one embodiment, at least one heating element 106 may further comprise a controller (including but not limited to a fuse, timer, thermostat, electronic circuit, microprocessor and etc.) configured to initiate or terminate heating of mask 100. The use of such a controller prevents at least one heating element 106 from continuously heating mask 100. The continued heating of mask 100 may result in 1) uncomfortable feeling to skin 2) heating of the mask to an undesired elevated temperature, and/or 3) unnecessary loss of battery power.

In one embodiment, the controller may further comprise a power switch. The power switch is operable to close the circuit between the battery and at least one heating element 106. In one embodiment, when the switch is positioned on the “off” configuration, the power to at least one heating element 106 is disconnected. In one embodiment, when the switch is positioned on the “on” configuration, the power to at least one heating element 106 is connected and heating of mask 100 is initiated.

In one embodiment, the controller may be positioned in breathing zone 108. In one embodiment, the controller may be positioned on either side of breathing zone 108. In one exemplary embodiment, the controller and the power source may be positioned on either side of breathing zone 108, when at least one heating element 106 is positioned within breathing zone 108. In one embodiment, the controller may be attached to the power source and positioned outside of mask 100. In one embodiment, controller may reside entirely on a single computing device, or may reside on a central server and run on any number of end-user devices via a communications network. The computing devices may include standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server, and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

The system software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text, or phone call, to alert a manager, expert, researcher, or other professional of the particular results.

Further embodiments of such mechanisms are described elsewhere herein or may be standard systems understood by those skilled in the art.

In one embodiment, at least one heating element 106 may be attached to outer layer 102 or inner layer 104 by any means known to one skilled in the art.

In one embodiment, one or more of outer layer 102, inner layer 104 and the at least one middle layer may further comprise a resealable slot or fold opening positioned within mask 100. The resealable slot or fold opening is configured to house at least one heating element 106 and prevent it from moving toward' s the wearer's skin. In one embodiment, the resealable slot or fold opening allows at least one heating element to be inserted, removed, and fixed in place during use.

In one embodiment, mask 100 may further comprise a temperature sensor. In one embodiment, the detected temperature in combination with a certain amount of time after achieving a certain detected temperature is used by the controller to cause heating element 106 to temporarily or permanently stop generating heat. In one embodiment, a timer may be used to signal to the controller to temporarily or permanently stop generating heat after a time of up to about 1-3 hours; however, it will be appreciated that the time period can be greater than 45 minutes.

In one embodiment, at least one heating element 106 may be removable. In one embodiment, at least one heating element 106 may be fixed in place. In one embodiment, mask 100 may be disposable. In one embodiment, mask 100 may be washable, after taking out at least one heating element 106. In one embodiment, mask 100 may be reusable.

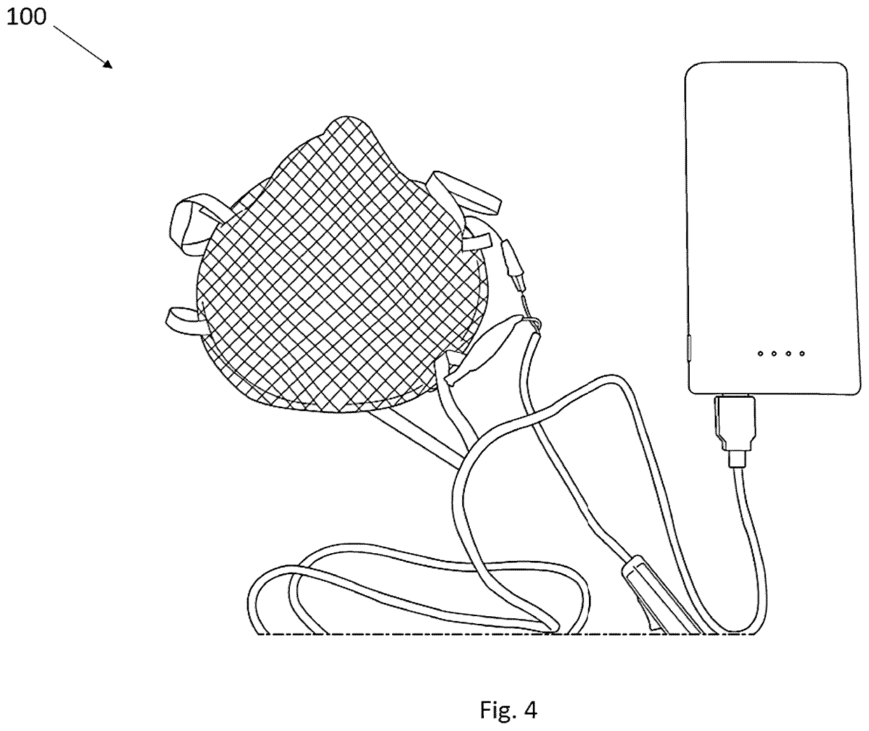

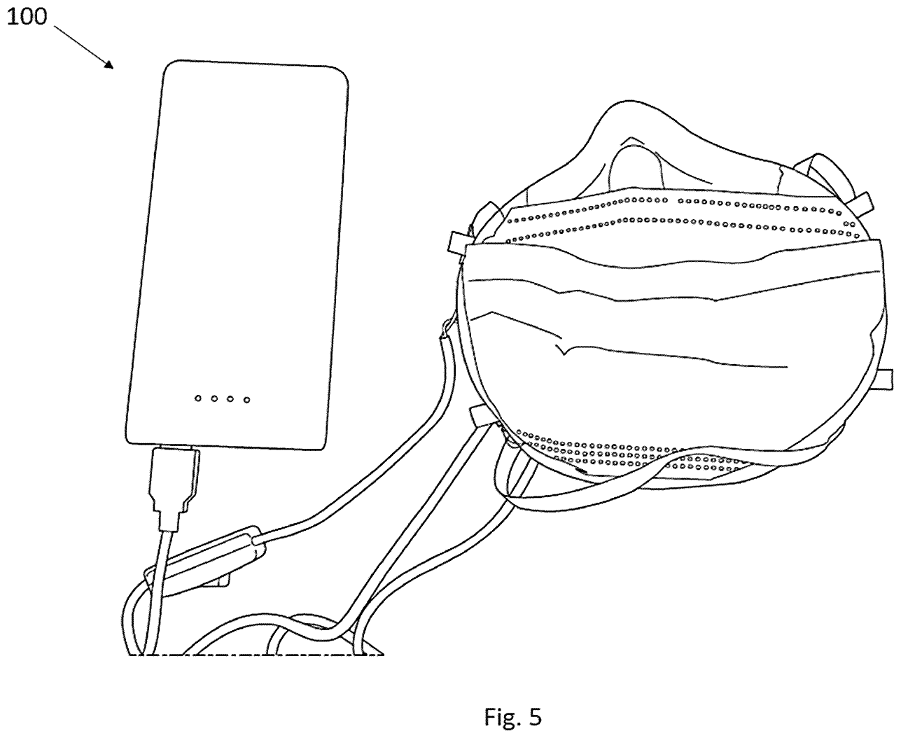

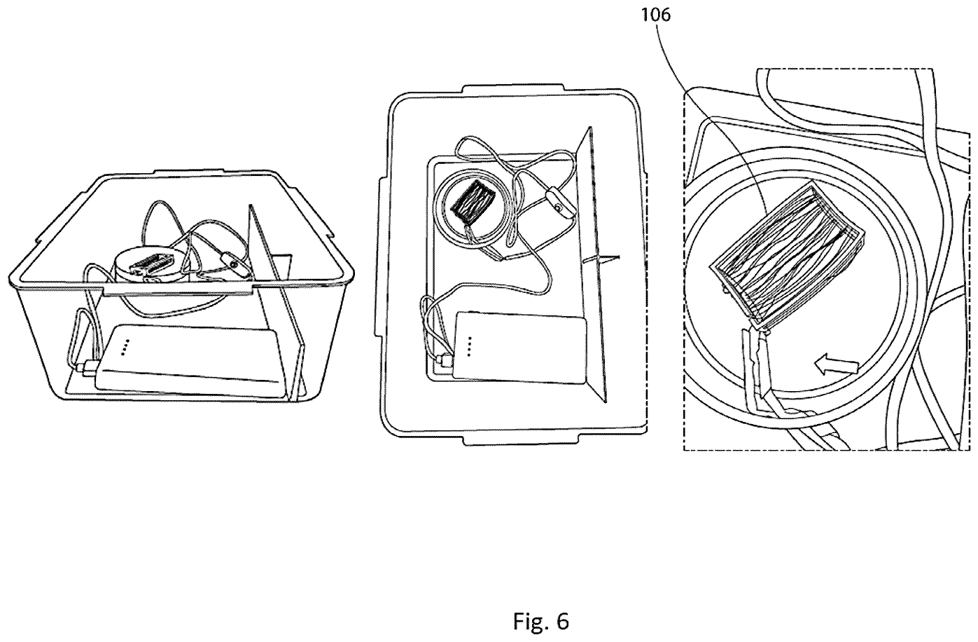

In one embodiment, at least one heating element 106 may be flexible. In one embodiment, at least one heating element 106 may be powered by a portable battery pack (FIG. 4, FIG. 5, and FIG. 6). In one embodiment, at least one heating element 106 may be powered by any other energy source including but not limited to a solar panel, fuel cell, etc. In one embodiment, at least one heating element 106 may be powered by a rechargeable source. In one exemplary embodiment, the rechargeable source may be a rechargeable battery pack normally used for charging cell phones. Embodiments comprising a rechargeable battery can further comprise one or more features to enable recharging, such as a cable port for connecting to a power source. In one embodiment, mask 100 may further comprise at least one plug positioned outside of mask 100, wherein the at least one plug is connected to at least one heating element 106 at one end and configured to allow attachment to a cable port connected to a power source at the other end.

In one embodiment, at least one heating element 106 may be powered by a replaceable battery. In one embodiment, the power source may be positioned within the mask in breathing zone 108. In one embodiment, the power source may be positioned within the mask on either sides of breathing zone 108. In one embodiment, at least a portion of the power source may be positioned within breathing zone 108. In one embodiment, the power source may be positioned external to mask 100. In one exemplary embodiment, the power source may be carried by the wearer. In one exemplary embodiment, the power source may be positioned within the wearer's pocket.

In one embodiment, at least one heating element 106 may further comprise a protective layer configured to insulate heating element from outer layer 102, inner layer 104 or the at least one middle layer. In one embodiment, any insulating material known to one skilled in the art may be used to cover at least one heating element 106.

In one embodiment, mask 100 may be secured to the wearer's head and face by any means known to one skilled in the art including but not limited to ear loops, head band, tie on straps, adhesive strips, and etc. In one embodiment, ear loops may comprise string like materials. In one embodiment, ear loops may be made from an elastic material. In one embodiment, the tie on straps may simply be four straps that are tied behind the head of the wearer. In one embodiment, the head band may comprise an elastic strap that is positioned behind the head. In one embodiment, a no-strap design may comprise non-irritant adhesive attached to the perimeter (a portion of the perimeter or the entire perimeter) of inner layer 104 that is attached to the skin of the wearer and provides an airtight seal that prevents air from leaking about the sides of mask 100.

In one embodiment, mask 100 may further comprise one or more deformable members. In one embodiment, the deformable members may be bent or pressed by a wearer into a configuration that allows the body of mask 100 to conform to a wearer's face. In one embodiment, the deformable member may assist in forming the seal between the sealing member and the wearer's face as it can maintain the sealing member in a desired configuration corresponding to the contours of the wearer's face. For example, the deformable member may be positioned at the upper part of the body of mask 100 corresponding to where a wearer's nose and cheeks would be positioned in use. In one embodiment, the deformable member may be positioned on outer layer 102, inner layer 104 or between inner layer 104 and the sealing member.

In one embodiment, the deformable member may be made of any deformable material that retains its shape after being bent or pressed by a wearer, such as a metal. In one embodiment, any material known to one skilled in the art may be used in the deformable member including, but not limited to, aluminum, steel, plastic, etc. In one embodiment, the deformable member may have a variety of shapes, sizes, and configurations that allow a wearer to bend or press it to conform the body of mask 100 to a wearer's face. For example, the deformable member may be straight, curved, angled, continuous, or discontinuous. In one embodiment, the deformable member may be attached to the body of mask 100 by any possible method including, but not limited to, heat welding, ultrasonic welding, and adhesive bonding.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention. The following working examples, therefore, specifically point out exemplary embodiments of the present invention, and are not to be construed as limiting in any way the remainder of the disclosure.

### Example 1: Functional Mask

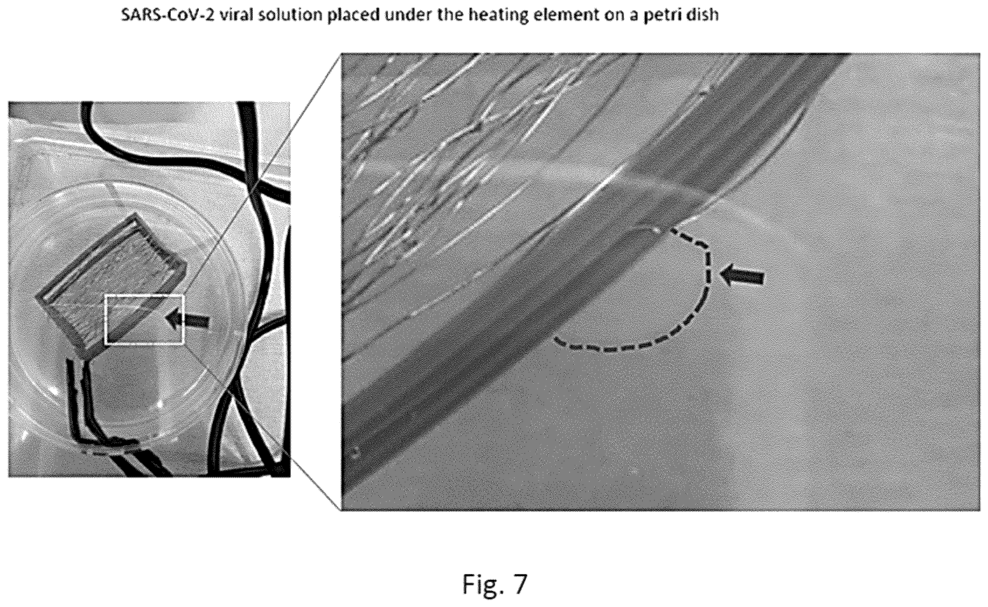

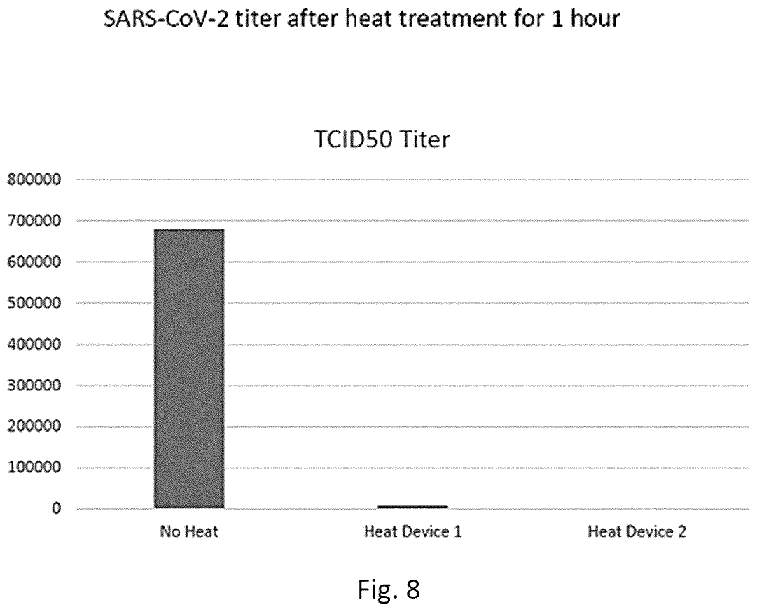

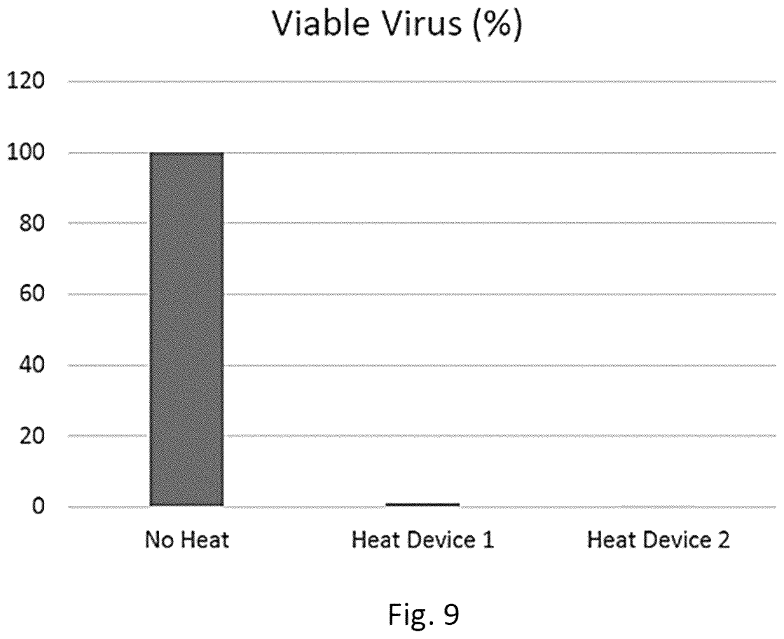

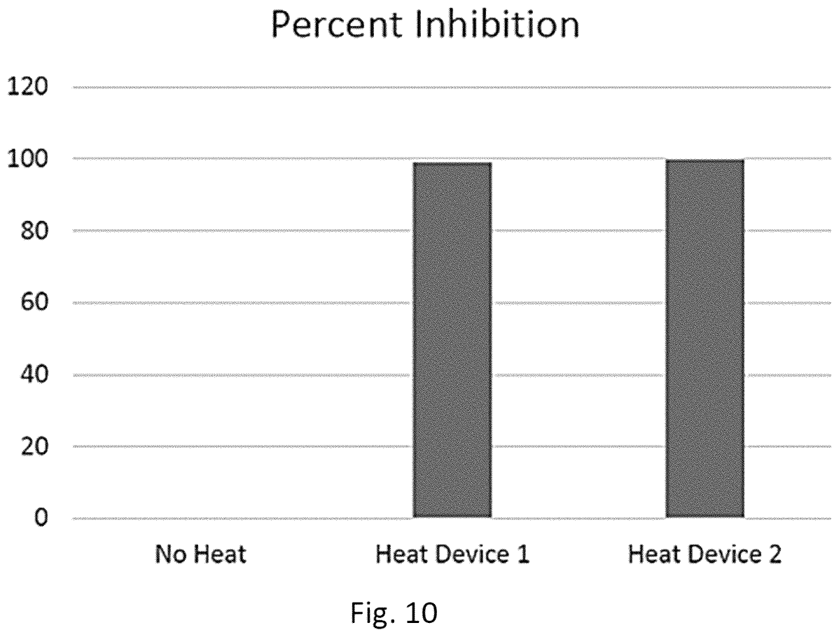

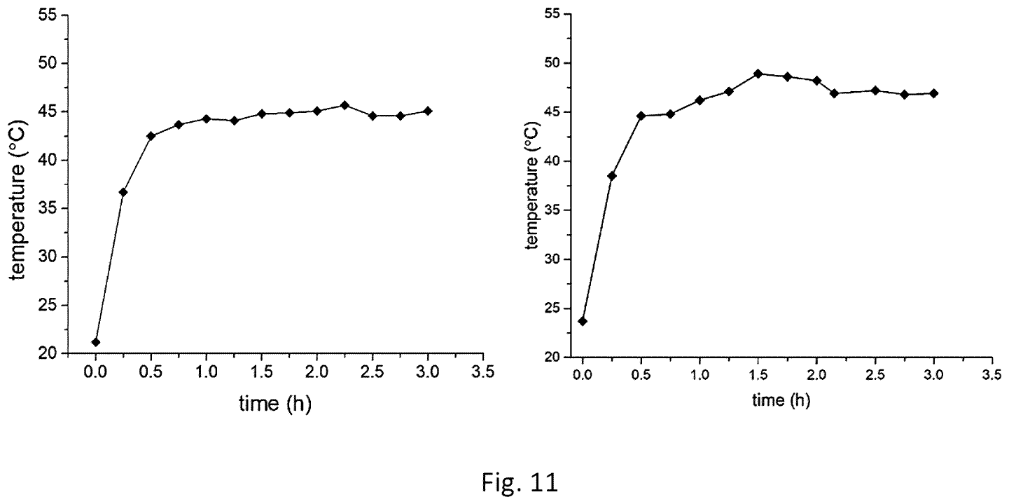

Referring now to FIG. 6 and FIG. 7, the result of an experiment investigating the effect of heat on inactivating viruses is shown. As shown in FIG. 6 and FIG. 7, heating elements were housed in a petri dish which was inoculated with SARS-CoV-2 (red arrow points to viral smear). SARS-COV-2 viral solution was placed under the heating element on a petri dish. Results show that 1 hour heat treatment inactivates virus over 99% (FIG. 8, FIG. 9, and FIG. 10). Further, temperature rise of the heating element was investigated, wherein the heating element was able to hold the temperature in a range between 40-50° C. for about 3 hours (FIG. 11).

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A face mask comprising:
an outer layer;
an inner layer; and
at least one heating element positioned between the outer layer and the inner layer.

2. The face mask of claim 1, wherein the mask further comprises at least one middle layer positioned between the outer layer and the inner layer.

3. The face mask of claim 1, wherein the outer layer further comprises a resealable slot or fold opening configured to allow insertion and removal of the at least one heating element.

4. The face mask of claim 1, wherein the inner layer further comprises a resealable slot or fold opening configured to allow insertion and removal of the at least one heating element.

5. The face mask of claim 1, wherein the inner layer and the outer layer both comprise a resealable slot or fold opening configured to allow insertion and removal of the at least one heating element.

6. The face mask of claim 1, wherein the mask is configured to cover a wearer's nasal and oral region.

7. The face mask of claim 1, wherein the at least one heating element is configured to heat up to a temperature ranging about 40-50° C.

8. The face mask of claim 1, wherein the at least one heating element further comprises a controller configured to initiate or terminate heating of at least one heating element.

9. The face mask of claim 1, wherein the at least one heating element is removable.

10. The face mask of claim 1, wherein the at least one heating element is powered by a battery.

11. The face mask of claim 1, wherein the at least one heating element is be powered by a rechargeable source.

12. The face mask of claim 11, wherein the rechargeable source is positioned outside the mask and is carried by a wearer.

13. The face mask of claim 1, wherein the at least one heating element is configured to allow passage of air through the at least one heating element to the inner layer and to a wearer.

14. The face mask of claim 1, wherein the at least one heating element is further covered by a protective insulating layer.

15. The face mask of claim 1, wherein the at least one heating element is an electrical heating element.

16. The face mask of claim 1, wherein the at least one heating element has a plurality of channels that allows air passage therethrough and wherein the plurality of channels have a parallel structure.

17. The face mask of claim 1, wherein the mask further comprises at least one strap configured to secure the mask to a wearer's face.

18. The face mask of claim 1, wherein the mask further comprises a deformable member, configured to be bent or pressed by a wearer into a configuration that allows the body of the mask to conform to the wearer's face.

19. The face mask of claim 1, wherein the mask further comprise a breathing zone positioned directly in front of the nasal and oral region.

20. The face mask of claim 19, wherein the at least one heating element is positioned within the breathing zone.
